(19) United States
(12) Patent Application Publication
(10) Pub. No.: US 2013/0129869 A1
(43) Pub. Date: May 23, 2013

(54) COMPOSITIONS COMPRISING A SHELF-LIFE STABILITY COMPONENT

(71) Applicants: Hooman Hafezi, Redwood City, CA (US); Raymond Schmidt, San Francisco, CA (US); Ai Ling Ching, San Francisco, CA (US)

(72) Inventors: Hooman Hafezi, Redwood City, CA (US); Raymond Schmidt, San Francisco, CA (US); Ai Ling Ching, San Francisco, CA (US)

(21) Appl. No.: 13/304,260

(22) Filed: Nov. 23, 2011

(51) Int. Cl. A23L 3/00

(52) U.S. Cl. 426/89; 426/654; 426/132; 426/321

(57) ABSTRACT

Compositions that include a shelf-life stability component are provided. In some instances, the compositions are ingestible compositions which include the shelf-life stability component and an ingestible component. Aspects of the invention further include methods of making and using the compositions.

A variety of different ingestible compositions have been developed for nutritional, therapeutic and non-therapeutic uses. Examples of different types of ingestible compositions include orally ingestible tablets, capsules and liquids. A given orally ingestible formulation may include a variety of different components, such as active agents, carrier materials (including binders, bulking agents and other excipients), flavoring agents, coloring agents, etc. More recently, ingestible compositions which include a device component, such as an RFID tag or an ingestible event marker, have been developed.

As with many consumer products, ingestible compositions are not manufactured at the time of and location of use. Instead, they are generally manufactured at one or more fabrication facilities, stored for a period of time and then shipped to the end-user. Upon receipt, the end-user may further store them for a period of time before use.

During the multiple storage periods, and even manufacturing periods, such as mentioned above, the quality of the ingestible composition, e.g., in terms of effectiveness, may be degraded in some way. For example, exposure to humidity, elevated temperatures, microorganisms and oxidizing agents, as well other environmental hazards, can negatively impact the quality of the ingestible composition. Shelf-life stability of ingestible compositions is therefore a significant consideration in their manufacture and use.

### SUMMARY

Compositions that include a shelf-life stability component are provided. In some instances, the compositions are ingestible compositions which include the shelf-life stability component and an ingestible component. Aspects of the invention further include methods of making and using the compositions.

### BRIEF DESCRIPTION OF THE FIGURES

FIGS. 1A and 1B provide side and top views, respectively, of one aspect of an ingestible event marker (IEM);

FIG. 2 provides a side view of one aspect of an ingestible composition that includes a mono-layer protective barrier;

FIG. 3 provides a side view of one aspect of an ingestible composition that includes a protective barrier made of a homogeneous blend of two different materials;

FIG. 4 provides a side view of one aspect of an ingestible composition that includes a protective barrier made of a heterogeneous structure of two different materials;

FIGS. 5A and 5B provide side views of one aspect of an ingestible composition that includes a multi-layer protective barrier;

FIG. 6 provides a side view of one aspect of an ingestible composition that includes a protective barrier made of an inter-digitated structure of two different materials;

FIG. 7 provides a side view of one aspect of an ingestible composition that includes a protective barrier made of an overlapping structure of two different materials;

FIG. 8 provides a side view of one aspect of an ingestible composition that includes a multi-layer protective barrier;

FIG. 9 provides a side view of one aspect of an ingestible composition that includes a multi-layer protective barrier; and

FIG. 10 provides a side view of one aspect of an ingestible composition that includes a galvanic protective barrier.

FIG. 11 provides a side view of one aspect of an ingestible composition that includes a mono-layer protective barrier with one or more fluid passageways.

### DETAILED DESCRIPTION

Compositions that include a shelf-life stability component are provided. In some instances, the compositions are ingestible compositions which include the shelf-life stability component and an ingestible component. Aspects of the invention further include methods of making and using the compositions.

### Compositions

Aspects of the invention include compositions having shelf-life stability component physically associated with a minimally dimensioned component. A shelf-life stability component is a component that imparts shelf-life stability to the composition, in that the shelf-life stability component enhances the storage stability of the composition by a quantifiable measure as compared to a control composition that lacks the shelf-life stability component. Shelf-life stability components of interest may enhance the shelf-life stability of the composition as compared to a suitable control by a magnitude of two-fold or greater, such as five-fold or greater including ten-fold or greater, e.g., twenty-five-fold or greater. The presence of the shelf-life stability component allows the composition to be stable for extended periods of time during or following manufacture, where the ingestible composition may be stable for one year or longer, such as two years or longer, including five years or longer, following manufacture when the composition maintained under conditions in which the temperature ranges from 10 to 40° C., the pressure ranges from 0.5 to 2.0 ATM and the relative humidity ranges from 10 to 100%. By “stable” is meant that the functionality of the composition does not degrade to a point that the composition is no longer suitable for use in its intended purpose. For example, if the composition includes a circuitry component, e.g., an ingestible event marker (such as described in greater detail below) or a micro-battery, the circuitry component continues to function for its intended purpose for the period of time between manufacture and ingestion when stored under the conditions described above. If the composition includes an active pharmaceutical agent, the amount of active agent following the storage time period may be 85% or more, such as 90% or more, including 95% or more of the original amount present in the composition following manufacture, e.g., as determined using an HPLC protocol or other suitable analytical technique which can distinguish the amount of active agent from any degradation byproducts, such as oxidation byproducts.

Minimally dimensioned components may vary in dimension, and in some instances have a longest dimension of 30 mm or less, such as 20 mm or less, e.g., 10 mm or less. The volume of these minimally dimensioned components of interest may also vary, where the volume in some instances may be 25 mm³ or less, such as 15 mm³ or less, including 10 mm³ or less. Of interest as minimally dimensioned components are components that are susceptible at least partial degradation during storage. Such components may or may not include circuitry component, e.g., as described in greater detail below. Compositions of interest that may include a shelf-life stability component include ingestible compositions, micro-batteries, etc.

### Ingestible Compositions

Aspects of the invention include ingestible compositions. In these instances, ingestible compositions of interest include both an ingestible component and shelf-life stability component. As the compositions are ingestible, they are configured to be ingested or swallowed, i.e., taken into the stomach by drawing through the throat and esophagus with a voluntary muscular action. Accordingly, the compositions are dimensioned so as to be capable of being ingested. In some instances, the compositions have a longest dimension of 30 mm or less, such as 20 mm or less, e.g., 10 mm or less. The volume of the ingestible composition may also vary so long as the composition is suitable for ingestion, where the volume in some instances may be 25 mm³ or less, such as 15 mm³ or less, including 10 mm³ or less.

The ingestible component is a portion or part of the ingestible composition that is configured for ingestion. The ingestible component may vary widely and may include one or more subcomponents, e.g., a pharmaceutically acceptable solid carrier (which may or may not include an active agent), a device (which may or may not include electronic circuitry), etc.

In some instances, the ingestible component includes a pharmaceutically acceptable solid carrier. Pharmaceutically acceptable solid carrier configurations include tablet and capsule configurations. While the pharmaceutically acceptable solid carrier may have a solid configuration, the solid configuration may include a liquid component, such as is found in a liquid capsule, which includes a liquid component present in a solid capsule. In some instances, the pharmaceutically acceptable solid carrier is configured to impart a controlled release profile to an active agent that is associated with the pharmaceutically acceptable solid carrier. Examples of pharmaceutically acceptable solid carriers of interest can be found in Remington's Pharmaceutical Sciences, Mace Publishing Company, Philadelphia, Pa., 17th ed. (1985).

Where desired, the pharmaceutically acceptable solid carrier may include an active agent. Active agents of interest include pharmaceutically active agents as well as non-pharmaceutical active agents, such as diagnostic agents. The phrase “pharmaceutically active agent” (also referred to herein as drugs) refers to a compound or mixture of compounds which produces a physiological result, e.g., a beneficial or useful result, upon contact with a living organism, e.g., a mammal, such as a human. Pharmaceutically active agents are distinguishable from such components as excipients, carriers, diluents, lubricants, binders and other formulating aids, and encapsulating or otherwise protective components. The pharmaceutically active agent may be any molecule, as well as binding portion or fragment thereof, that is capable of modulating a biological process in a living subject. In certain aspects, the pharmaceutically active agent may be a substance used in the diagnosis, treatment, or prevention of a disease or as a component of a medication. The pharmaceutically active agent is capable of interacting with a target in a living subject. The target may be a number of different types of naturally occurring structures, where targets of interest include both intracellular and extracellular targets. Such targets may be proteins, phospholipids, nucleic acids and the like, where proteins are of particular interest. Specific proteinaceous targets of interest include, without limitation, enzymes, e.g., kinases, phosphatases, reductases, cyclooxygenases, proteases and the like, targets comprising domains involved in protein-protein interactions, such as the SH2, SH3, PTB and PDZ domains, structural proteins, e.g., actin, tubulin, etc., membrane receptors, immunoglobulins, e.g., IgE, cell adhesion receptors, such as integrins, etc., ion channels, transmembrane pumps, transcription factors, signaling proteins, and the like. Broad categories of active agents of interest include, but are not limited to: cardiovascular agents; pain-relief agents, e.g., analgesics, anesthetics, anti-inflammatory agents, etc.; nerve-acting agents; chemotherapeutic (e.g., anti-neoplastic) agents; neurological agents, e.g., anti-convulsants, etc. The amount of active agent that is present in the solid carrier may vary. In some instances, the amount of active agent that is present may range from 0.01 to 100% by weight.

Further examples of pharmaceutically acceptable solid carriers and active agents which may or may not be included therein are described in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; PCT application serial no. PCT/US2008/077753 published as WO2009/042812; PCT application serial no. PCT/US2008/085048 published as WO2009/070773; PCT application serial no. PCT/US2009/36231 published as WO2009/111664; PCT application serial no. PCT/US2009/049618 published as WO2010/005877; PCT application serial no. PCT/US2009/053721 published as WO2010/019778; PCT application serial no. PCT/US2009/060713 published as WO2010/045385; PCT application serial no. PCT/US2009/064472 published as WO2010/057049; PCT application serial no. PCT/US2009/067584 published as WO2010/068818; PCT application serial no. PCT/US2009/068128 published as WO2010/075115; PCT application serial no. PCT/US2010/020142 published as WO2010/080765; PCT application serial no. PCT/US2010/020140 published as WO2010/080764; PCT application serial no. PCT/US2010/020269 published as WO2010/080843; PCT application serial no. PCT/US2010/028518 published as WO2010/111403; PCT application serial no. PCT/US2010/032590 published as WO2010/129288; PCT application serial no. PCT/US2010/034186 published as WO2010/132331; PCT application serial no. PCT/US2010/055522 published as WO2011/057024; the disclosures of which are herein incorporated by reference.

In addition to or instead of a pharmaceutically acceptable solid carrier, ingestible compositions may include a device. The term “device” is used broadly to refer to a mechanical and/or electrical component configured for a particular purpose, where the device may or may not include a circuitry component.

Of interest as devices are ingestible devices, e.g., RFID-enabled devices; ingestible event markers, etc. An ingestible event marker (IEM) is a device that is dimensioned to be ingestible and includes an identifier circuitry component and, optionally, a current path extender, e.g., a membrane, sometimes referred to herein as a “skirt”. To illustrate, various aspects of an IEM may include a control device for altering conductance; and a partial power source. The partial power source may include a first material electrically coupled to the control device; and a second material electrically coupled to the control device and electrically isolated from the first material.

Upon ingestion, the IEM contacts a conducting fluid, e.g., stomach fluid. When the IEM is in contact with the conducting liquid, a current path is formed through the conducting liquid between the first and second materials. The voltage potential created between the materials provides the power for operating the IEM as well as produces the current flow through the conducting fluid and the system. In one aspect, the IEM operates in direct current mode. In an alternative aspect, the IEM controls the direction of the current so that the direction of current is reversed in a cyclic manner, similar to alternating current. The current path through the system is controlled by the control device. Completion of the current path allows for the current to flow and in turn a receiver, not shown, can detect the presence of the current and recognize that the system has been activated and the desired event is occurring or has occurred.

In one aspect, the two materials are similar in function to the two electrodes needed for a direct current power source, such as a battery. The conducting liquid acts as the electrolyte needed to complete the power source. The completed power source described is defined by the electrochemical reaction between the materials of the IEM and enabled by the fluids of the body. The completed power source may be viewed as a power source that exploits electrochemical conduction in an ionic or a conducting solution such as gastric fluid, blood, or other bodily fluids and some tissues.

In certain aspects, the complete power source or supply is one that is made up of active electrode materials, electrolytes, and inactive materials, such as current collectors, packaging, etc. The active materials are any pair of materials with different electrochemical potentials. Suitable materials are not restricted to metals, and in certain aspects the paired materials are chosen from metals and non-metals, e.g., a pair made up of a metal (such as Mg) and a salt (such as CuI). With respect to the active electrode materials, any pairing of substances—metals, salts, or intercalation compounds—with suitably different electrochemical potentials (voltage) and low interfacial resistance are suitable. Where desired, the voltage provided by the two dissimilar electrochemical materials upon contact of the materials of the power source with the target physiological site is 0.001 V or higher, including 0.01 V or higher, such as 0.1 V or higher, e.g., 0.3 V or higher, including 0.5 volts or higher, and including 1.0 volts or higher, where in certain aspects, the voltage ranges from about 0.001 to about 10 volts, such as from about 0.01 to about 10 V.

Anode materials of interest include, but are not limited to: magnesium, zinc, sodium, lithium, iron and alloys thereof, e.g., Al and Zn alloys of Mg, which may or may not be intercalated with a variety of materials such, as graphite with Li, K, Ca, Na, Mg, and the like. Cathode materials of interest include, but are not limited to, copper salts, such as copper salts of iodide, chloride, bromide, sulfate, formate, Fe³⁺ salts, e.g., orthophosphate, pyrophosphate, etc. One or both of the metals may be doped with a non-metal, for example to enhance the voltage output of the battery. Non-metals that may be used as doping agents in certain aspects include, but are not limited to: sulfur, iodine and the like. In certain aspects, the electrode materials are cuprous iodine (CuI) or cuprous chloride (CuCl) as the anode and magnesium (Mg) metal or magnesium alloy as the cathode. Aspects of the present invention use electrode materials that are not harmful to the human body.

With respect to current signatures, the current signatures may distinguish one class of ingestible event marker from other types or may be universally unique, such as where the current signature is analogous to a human fingerprint which is distinct from any other fingerprint of any other individual and therefore uniquely identifies an individual on a universal level. In various aspects, the control circuit may generate a variety of different types of communications, including but not limited to: RF signals, magnetic signals, conductive (near-field) signals, acoustic signals, etc.

In various aspects, the IEM may further comprise a current path extender such as a membrane which, for example, produces a virtual dipole length between the pair of transmission elements that is larger than the actual dipole length. In addition to controlling the magnitude of the current path between the materials, a membrane (sometimes referred to herein as “amplifier” or “skirt”) is used to increase the “length” of the current path and, hence, act to boost the conductance path, as disclosed in the U.S. patent application Ser. No. 12/238,345 entitled, “In-Body Device with Virtual Dipole Signal Amplification” filed Sep. 25, 2008, and in the U.S. Pat. No. 7,978,064 entitled, “Communication System with Partial Power Source” dated Jul. 12, 2011 the entire content of which are incorporated herein by reference.

Receivers, sometimes referred to herein as a “detector” may detect the communication, e.g., current. Receivers may not require any additional cable or hard wire connection between the device and a receiver of the communication, sometimes referred to herein as a detector.

In the ingestible composition of interest, the IEM may be stably associated in some manner to another ingestible component, e.g., pharmaceutically acceptable carrier component (e.g., as described above). By “stably associated” is meant that the IEM and second ingestible component, e.g., a pharmaceutically acceptable carrier component, do not separate from each other, at least until administered to the subject in need thereof, e.g., by ingestion. As the IEMs are dimensioned to be ingestible, they are sized so that they can be placed in a mammalian, e.g., human or animal, mouth and swallowed. In some instances, IEMs of the invention have a longest dimension that is 30 mm or less, such as 20 mm or less, including 5 mm or less.

Various aspects of ingestible event markers of interest (including protocols for the fabrication thereof) are described in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; PCT application serial no. PCT/US2008/077753 published as WO2009/042812; PCT application serial no. PCT/US2008/085048 published as WO2009/070773; PCT application serial no. PCT/US2009/36231 published as WO2009/111664; PCT application serial no. PCT/US2009/049618 published as WO2010/005877; PCT application serial no. PCT/US2009/053721 published as WO2010/019778; PCT application serial no. PCT/US2009/060713 published as WO2010/045385; PCT application serial no. PCT/US2009/064472 published as WO2010/057049; PCT application serial no. PCT/US2009/067584 published as WO2010/068818; PCT application serial no. PCT/US2009/068128 published as WO2010/075115; PCT application serial no. PCT/US2010/020142 published as WO2010/080765; PCT application serial no. PCT/US2010/020140 published as WO2010/080764; PCT application serial no. PCT/US2010/020269 published as WO2010/080843; PCT application serial no. PCT/US2010/028518 published as WO2010/111403; PCT application serial no. PCT/US2010/032590 published as WO2010/129288; PCT application serial no. PCT/US2010/034186 published as WO2010/132331; PCT application serial no. PCT/US2010/055522 published as WO2011/057024; the disclosures of which are herein incorporated by reference.

In certain aspects, the ingestible event markers are disrupted upon administration to a subject. As such, in certain aspects, the compositions are physically broken, e.g., dissolved, degraded, eroded, etc., following delivery to a body, e.g., via ingestion, injection, etc. The compositions of these aspects are distinguished from devices that are configured to be ingested and survive transit through the gastrointestinal tract substantially, if not completely, intact.

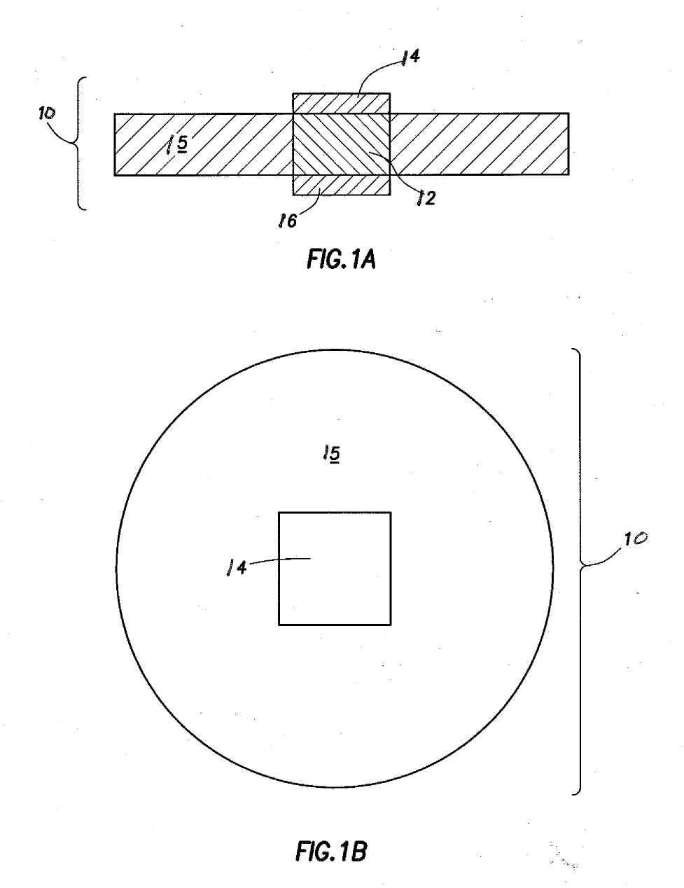

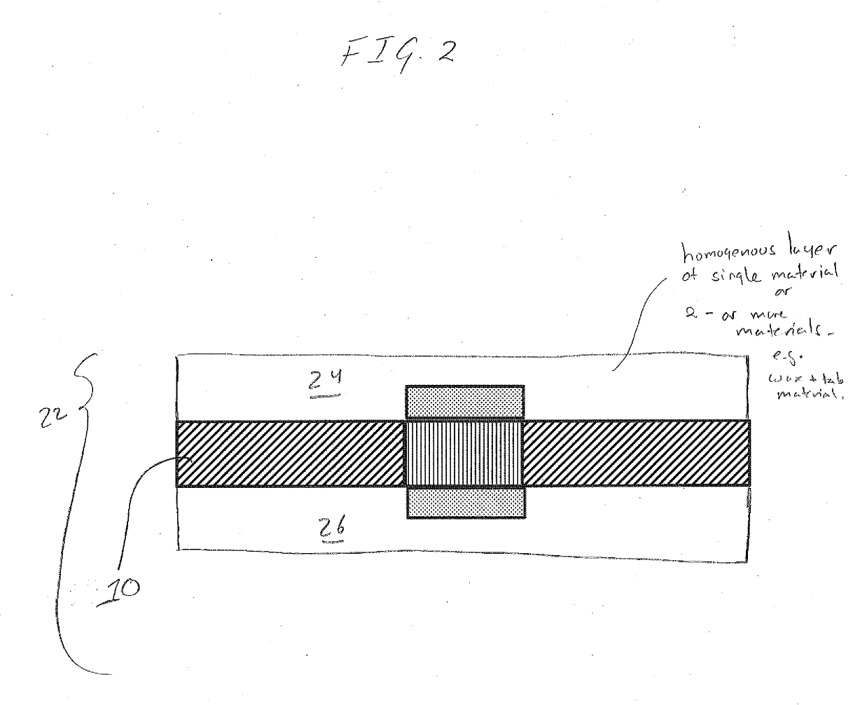

FIG. 1A provides a view of an aspect of an IEM of interest which has a current extender in the form of a membrane that extends beyond the outer edges of the signal transmission elements to provide a virtual dipole having a length that is longer than the actual dipole between the signal transmission elements. As shown in FIG. 1A, IEM 10 includes integrated circuit 12, having a first electrochemical material 14 (which may comprise two distinct material layers) and a second electrochemical material 16. Also shown is disc shaped membrane 15. FIG. 1B provides an overhead view of the IEM shown in FIG. 1A, showing the disc shape of first electrochemical material 14 and the positioning of the first electrochemical material in the center of disc shaped membrane 15. The distance that the edge of the membrane may extend beyond the edge of electrodes may vary, and in certain aspects is 0.05 mm or more, e.g., 0.1 mm or more, including 1.0 mm or more, such as 5.0 mm or more and including 10 mm or more, where the distance may not exceed 100 mm in certain aspects.

As can be seen in the aspect depicted in FIGS. 1A to 1B, the first and second electrochemical materials may have any convenient shape, e.g., square, disc, etc. The disc shaped membrane 15 is a planar disc structure, where the edge of the membrane extends beyond the edge of the first and second electrochemical materials. In the depicted aspect, the radius of the membrane is longer than the radius of the first and second electrochemical materials, e.g., by 1 mm or more, such as by 10 mm or more.

Membranes may have “two-dimensional” or “three-dimensional” configurations, as desired. Membrane configurations of interest are further described in PCT application serial no. US20081077753 published as WO2009/042812, PCT application serial no. US2010/020142 published as WO2010/080765 as well as PCT application serial no. US2010/032590 published as WO2010/129288; the disclosures of which are herein incorporated by reference.

The membrane may be fabricated from a number of different materials, where the membrane may be made of a single material or be a composite of two or more different types of materials, as developed in greater detail below. In certain instances, the membrane will have a mechanical strength sufficient to withstand the mechanical forces typical of the gastrointestinal (GI) tract without folding onto itself and losing its shape. This desired mechanical strength may be chosen to last for at least the duration of the communication, which may be 1 second or longer, such as at least 1 minute or longer, up to 6 hours or longer. In certain aspects, the desired mechanical strength is selected to least for a period of time ranging from 1 to 30 minutes. The desired mechanical strength can be achieved by proper selection of polymer and/or fillers, or mechanical design (e.g., lamination of multiple layers, or curvature of the amplifier surface) to increase the mechanical strength of the final structure.

Membranes of the invention are ones that are electrically insulating. As such, the materials from which the membranes are fabricated are electrically insulating materials. A given material is electrically insulating if it has a resistivity that is two times or greater than the medium in which the device operates, e.g., stomach fluid, such as ten times or greater, including 100 times or greater than the medium in which the device operates.

Where desired, an active agent (e.g., as described above) may be present in one or more of the IEM components, e.g., in the electrochemical materials, the support, the membrane, etc. Examples of such configurations are described in PCT application serial no. US2010/032590 published as WO2010/129288; the disclosures of which are herein incorporated by reference.

### Other Minimally Dimensioned Components

Aspects of the invention further include compositions that are not necessarily ingestible. As summarized above, such compositions may include a shelf-life stability components (e.g., as summarized above and described in greater detail below) physically associated with a minimally dimensioned component. While the minimally dimensioned component may vary, e.g., as described above, in some instances the minimally dimensioned component is a micro-battery. Micro-batteries of interest may include “all-solid” batteries, and may include components of a battery, such as current collectors, positive and negative electrodes, an electrolyte, in a minimally dimensioned structure, e.g., as described above. In some instances, micro-batteries of interest are thin films, which may be obtained by deposition, such as by physical vapor deposition (PVD) or chemical vapor deposition (CVD). The micro-battery may take a variety of different configurations, such as but not limited to: a chip configuration, a cylinder configuration, a spherical configuration, a disc configuration, etc., where a particular configuration may be selected based on intended application, method of manufacture, etc. In certain embodiments, the mciro-battery is dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm. In certain embodiments the micro-battery is 1 mm³ or smaller, such as 0.1 mm³ or smaller, including 0.2 mm³ or smaller.

### Shelf-Life Stability Component

As summarized above, an aspect of compositions of interest is a shelf-life stability component. Shelf-life stability components are elements of the compositions that enhance shelf-life stability of the composition as compared to a suitable control, e.g., as described above. Shelf-life stability components may vary widely, and may or may not be integrated with one or more other components of the compositions, e.g., a pharmaceutically acceptable solid carrier, an ingestible event marker, a micro-battery, etc. Furthermore, a given composition may include a single shelf-life stability component or two or more distinct shelf-life stability components, as desired. Examples of different types of shelf-life stability components of interest include, but are not limited to: a water vapor desensitizer (e.g., a protective barrier, a desiccant, etc.), an electrochemical material variant that imparts shelf-life stability, an antioxidant, a stabilizer, or combination thereof, etc.

Of interest as shelf-life stability components are water vapor desensitizers. Water vapor desensitizers are components that reduce the sensitivity of the ingestible component or portions thereof to the deleterious effects of water vapor which may be present in the environment of the ingestible composition. Deleterious effects are harmful results of exposure to water vapor, where examples of such effects include loss or chemical change of material, color change, loss of performance, etc. The magnitude of deleterious effect reduction may vary, and may be 5% or greater, such as 10% or greater, including 25% or greater. The particular protocol for determining such magnitude may vary depending on the particular deleterious effect of interest. Water vapor desensitizers of interest include, but are not limited to: protective barriers, water vapor sequestering agents, etc.

In some instances, the water vapor desensitizer is a protective barrier. Protective barriers of interest include any structure or element that functions as an obstruction, hindrance, or impediment to the passage of water vapor from one portion of the ingestible composition to another, e.g., from the exterior of the ingestible composition to another region of the ingestible composition, e.g., an interior location that houses an IEM. Of interest as protective barriers are those barriers that rapidly disrupt upon contact with a liquid, such as an aqueous liquid, e.g., stomach acid. By “rapidly disrupt” is meant that, upon contact with the liquid, the barrier is compromised in some fashion, such that it ceases to function as a complete barrier in a limited period of time, e.g., 60 minutes or less, such as 15 minutes or less, including 2 minutes or less. The protective barrier may be disrupted according to a number of different mechanisms, such as physical disruption, dissolution, etc.

Protective barriers may enclose an entire ingestible composition or a component thereof (e.g., an IEM) or be present on just a portion (e.g., one or more surfaces) of an ingestible composition or component thereof, as desired. The dimensions of a given barrier may vary, and in some instances the barrier has a thickness of 10 μm or greater, such as 25 μm or greater, including 50 μm or greater. In some instances, the thickness ranges from 10 to 1000 μm, such as 25 to 500 μm including 50 to 200 μm. Protective barriers may have a variety of different configurations, ranging from homogenous layers of a single material to heterogeneous layers of two or more materials to multilayer structures of two or more materials. Examples of various types of protective barriers of interest are now described in greater detail.

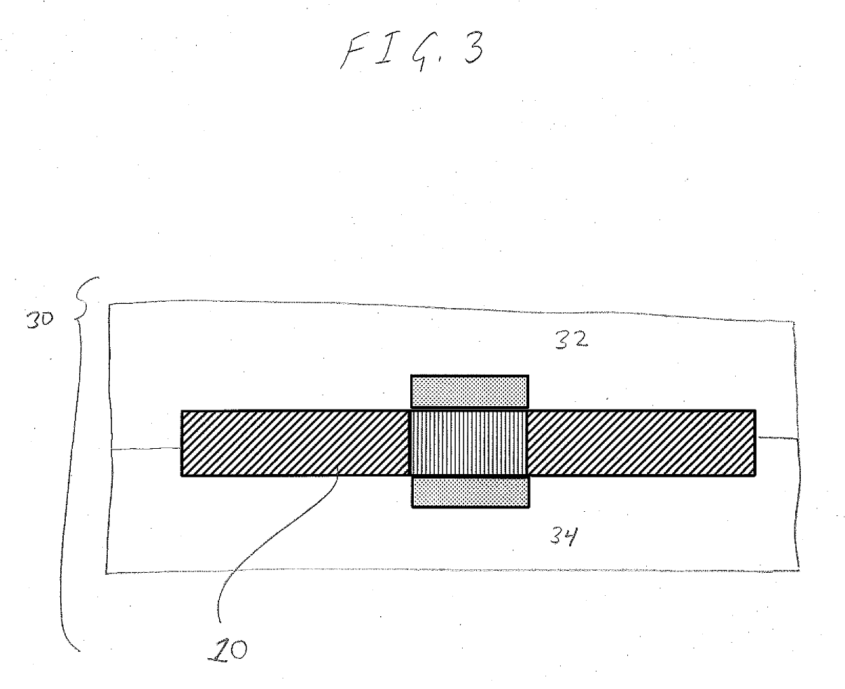

FIG. 2 provides a side view of an ingestible composition which includes a mono-layer protective barrier made of a single material and an IEM device. In FIG. 2, ingestible composition 22 includes an IEM component 10, e.g., as described in FIGS. 1A and 1B, and first and second protective barriers, 24 and 26, present on opposing sides of the IEM and each in the form of a single homogenous layer. The thickness of each protective barrier may vary, where in some instances the thickness ranges from 25 to 500 μm including 50 to 200 μm. Each protective barrier may include a single material, or be a homogeneous mixture of two or more different materials, as reviewed in greater detail below.

A variety of different materials may be employed in protective barrier 24, where materials of interest are those that impart hydrophobicity to the layer such that the layer acts as a suitable water vapor desensitizer. In addition to acting as a water vapor barrier prior to contact with a liquid, the protective barrier will also be made up of a material that imparts the desired rapid disruptability to the protective barrier upon contact of the protective barrier with a liquid.

Materials of interest include, but are not limited to, lipids and functionally analogous materials which are solid at room temperature, are suitable for ingestion, are non-toxic and dissociate from each other (e.g., melt or dissolve) at internal body temperatures (i.e., core body temperatures, where such materials may be referred to as low-melting point materials). Lipids of interest include fatty acyls, glycerolipids, glycerophospholipids, etc. Lipid materials that find use in protective barriers include, but are not limited to: long chain organic materials, e.g., waxes, such as acrawax, bayberry wax, beeswax, candelilla wax, castor wax, carnauba wax, ceresin wax, coconut oil, cotton seed oil, esparto wax, glycowax, jojoba wax, Japan wax, lignite wax, linear polyethylene wax, microcrystalline petroleum wax, montan wax, olive oil, ouricouri wax, ozokerite wax, paraffin wax, rice bran wax, shellac wax, silicone waxes, synthetic waxes, sugarcane wax, cetyl palmitate, etc.; fatty alcohols, e.g., cetyl alcohol, lanolin alcohol, stearyl alcohol, etc.; fatty acids, such as lauric acid, myristic acid, palmitic acid, stearic acid, behenic acid, lignoceric acid, ceratic acid, montanoic acid, isostearic acid, isononanoic acid, 2-ethylhexanoic acid, oleic acid, ricinoleic acid, linoleic acid, linolenic acid, erucic acid, soybean fatty acid, linseed fatty acid, dehydrated castor fatty acid, tall oil fatty acid, tung oil fatty acid, sunflower fatty acid, safflower fatty acid, etc.; phospholipids; and triglycerides, etc.

Protective barriers of interest may further include pharmaceutically acceptable polymeric materials, including but not limited to, cellulosic materials, such as ethyl cellulose, cellulose acetate phthalate, cellulose acetate trimaletate, hydroxy propyl methylcellulose phthalate, polyvinyl acetate phthalate, polyvinyl alcohol phthalate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, ethyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, pullulan, collagen, casein, agar, gum arabic, sodium carboxymethyl cellulose, (swellable hydrophilic polymers) poly(hydroxyalkyl methacrylate) (molecular weight 5 k to 5000 k), polyvinylpyrrolidone (molecular weight 10 k to 360 k), anionic and cationic hydrogels, zein, polyvinyl alcohol having a low acetate residual, a swellable mixture of agar and carboxymethyl cellulose, copolymers of maleic anhydride and styrene, ethylene, propylene or isobutylene, pectin (molecular weight 30 k to 300 k), polysaccharides such as agar, acacia, karaya, tragacanth, algins and guar, polyethylene oxides (molecular weight 100 k to 5000 k), diesters of polyglucan, crosslinked polyvinyl alcohol and poly N-vinyl-2-pyrrolidone, hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, methyl ethyl cellulose, ethylhydroxy ethylcellulose, cellulose acetate, cellulose butyrate, cellulose propionate, gelatin, starch, maltodextrin, pullulan, polyvinyl pyrrolidone, polyvinyl alcohol, polyvinyl acetate, glycerol fatty acid esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof, pharmaceutically acceptable acrylic polymers, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers, etc.;

Also of interest as materials for protective barriers are ingestible metallic materials, e.g., gold, silver, titanium, copper, iron, magnesium, etc, as well as combinations thereof (see e.g., the galvanic protective layers described in greater detail below). Also of interest as materials for protective barriers are carbon allotropes having the desired properties, such as graphite, amorphous carbon, etc.

While the protective layer may be made up of a single type of material, in some instances the protective layer may be a homogenous blend (i.e., uniform mixture) of two or more different materials, where the second material may or may not be a material such as listed above, or another type of material which desirably modifies the properties of the first material. By homogeneous blend is meant a uniform mixture of the two or more materials. Accordingly, the protective barrier will not include regions or domains of a substantial volume that include only type of material to the exclusion of the other. When present, the weight ratio of first to second material may vary, and in some instances will range from 1% to 99%, such as 25% to 75% and including 25% to 35%.

In some instances, the second material may enhance disruptability of the layer upon contact with a liquid, as desired, where the particular mechanism by disruptability is enhanced may vary. For example, the second material may be a solubilizing agent that enhances solubility of the layer, such that the two or more distinct materials making up the protective barrier include a first material and a second material that solubilizes the first material. Solubilizing agents of interest include, but are not limited to, emulsifiers (e.g., surfactants), enzymes, pH sensitive materials, etc. Surfactants of interest include pharmaceutically acceptable anionic surfactants, cationic surfactants, amphoteric (amphipathic/amphiphilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, and alkyl sulfates. Suitable pharmaceutically acceptable non-ionic surfactants include, for example, polyoxyethylene compounds, lecithin, ethoxylated alcohols, ethoxylated esters, ethoxylated amides, polyoxypropylene compounds, propoxylate alcohols, ethoxylated/propoxylated block polymers, and propoxylated esters, alkanolamides, amine oxides, fatty acid esters of polyhydric alcohols, ethylene glycol esters, diethylene glycol esters, propylene glycol esters, glyceryl esters, polyglyceryl fatty acid esters, SPAN's (e.g., sorbitan esters), TWEEN's sucrose esters, and glucose (dextrose) esters. Other suitable pharmaceutically acceptable surfactants/co-solvents (solubilizing) agents include acacia, benzalkonium chloride, cholesterol, emulsifying wax, docusate sodium, glyceryl monostearate, lanolin alcohols, lecithin, poloxamer, poloxytheylene castor oil derivatives, poloxyethylene sorbitan fatty acid esters, poloxyethylene stearates, sodium lauryl sulfates, sorbitan esters, stearic acid, and triethanolamine. Mixed surfactant/wetting agent systems are also useful in conjunction with the present invention. Examples of such mixed systems include, for example, sodium lauryl sulfate/polyethylene glycol (PEG) 6000 and sodium lauryl sulfate/PEG 6000/stearic acid. Enzymes may also find use as solubilizers, such as where the first material is a substrate for the enzyme. Examples of enzymes of interest include, but are not limited to hydrolases, e.g., esterases; oxidoreductases, etc. Also of interest are pH sensitive materials, in which the material is insoluble/impenetrable during storage, but soluble at low pH, e.g., a pH less than 6, such as a pH less than 5. Examples of such materials include, but are not limited to: methacrylate and methacrylic acids, such as EPO (cationic copolymer based on dimethylaminoethyl methacrylate, butyl methacrylate, and methyl methacrylate), etc. Also of interest as solubilizing materials are materials that generate heat upon contact with an aqueous solution, such as stomach fluid, e.g., where such materials may increase the rate at which the protective material melts. Examples of such materials include, but are not limited to: salts with high enthalpy of solution, e.g., magnesium sulfate, calcium chloride, etc.

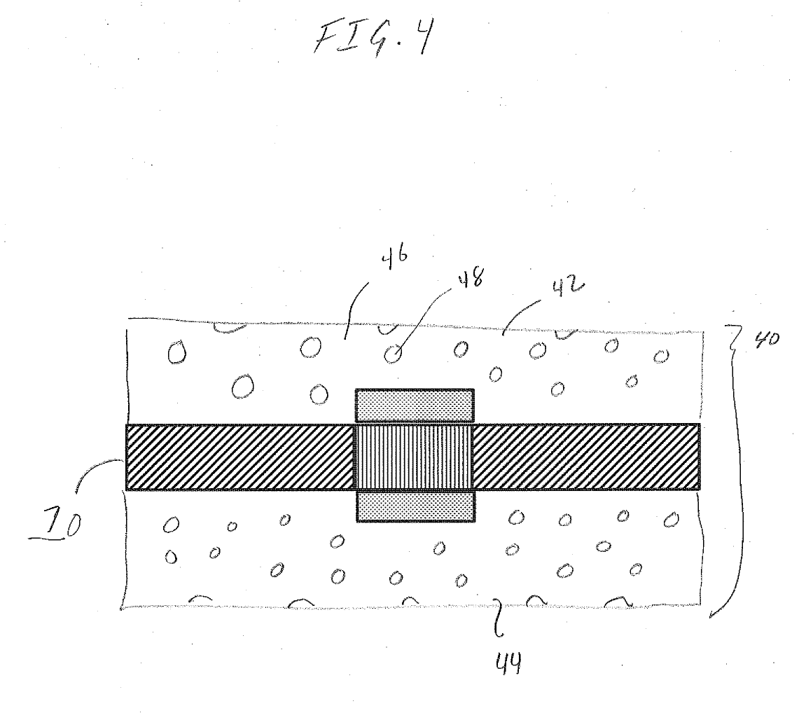

One type of protective barrier of interest that includes two or more different materials is a protective barrier that is made up of a pharmaceutical tablet carrier material and a barrier material, e.g., as illustrated in FIG. 3. In FIG. 3, ingestible composition 30 includes IEM device 10 which is sandwiched between first and second tablet halves 32 and 34 such that the ingestible composition 30 is in the form of a tablet. Each tablet half 32 and 34 includes a fused blend of a first tablet carrier material and a second protective barrier material. In the configuration shown in FIG. 3, the protective barrier material is present throughout each tablet half 32 and 34. An alternative configuration of interest is one which only an outer coating of surrounding the ingestible composition is made up of the blend of a carrier material and second protective barrier material. In such instances, the coating may be any convenient thickness, e.g., 100μ or thinner, such as 10μ or thinner, including 1μ or thinner.

The first tablet carrier material is made of one or more pharmaceutically acceptable tablet excipient materials. Tablet carrier materials of interest include, but are not limited to: fillers or extenders, such as starches, lactose, sucrose, glucose, mannitol, and/or silicic acid, talc; binders such as carboxymethylcellulose, ethyl cellulose and cellulose acetate, alginates, gelatin, polyvinyl pyrrolidone, sucrose and/or acacia; humectants such as glycerol; disintegrating agents such as agar-agar, calcium carbonate, potato or tapioca starch, alginic acid, silicates, and/or sodium carbonate; solution retarding agents such as paraffin; absorption accelerators such as quaternary ammonium compounds; wetting agents such as cetyl alcohol and/or glycerol monostearate; absorbents such as kaolin and/or bentonite clay; lubricants such as talc, calcium stearate, magnesium stearate, solid polyethylene glycols, sodium lauryl sulfate, and/or mixtures thereof; coloring agents; and buffering agents. Antioxidants can also be present in the pharmaceutical compositions of the invention. Examples of pharmaceutically acceptable antioxidants include: water-soluble antioxidants such as ascorbic acid, cysteine hydrochloride, sodium bisulfate, sodium metabisulfate sodium sulfite and the like; oil-soluble antioxidants such as ascorbyl palmitate, butylated hydroxyanisole (BHA), butylated hydroxytoluene (BHT), lecithin, propyl gallate, alpha-tocopherol, and the like; and metal-chelating agents such as citric acid, ethylenediamine tetraacetic acid (EDTA), sorbitol, tartaric acid, phosphoric acid, and the like.

The second protective barrier material may be a material made up of one or more ingredients, where the material melts at an elevated temperature in a manner that causes the second material to fill void spaces, e.g., pores, in the first carrier component. The elevated temperature at which the second material melts is one at which the first material does not physically change and one at which the components of the ingestible composition, e.g., the IEM device, are not damaged. In some instances, the elevated temperature at which the protective barrier material melts ranges from 25° C. to 160° C., such as 80° C. to 120° C. Any convenient material may be selected as the second material, where materials of interest include, but are not limited to: lipid materials (e.g., as described above), waxes, oils, and the like.

Ingestible compositions as shown in FIG. 3 may be produced using any convenient protocol. In some instances, a variation of the IEM tablet production protocols disclosed in PCT Application Serial No. PCT/US2006/016370 published as WO2006/116718; PCT Application Serial No. PCT/US2010/020142 published as WO2010/080765 and PCT Application Serial No. PCT/US2010/034186 published as 2010/132331 (the disclosures of which applications are incorporated by reference) is employed. In the variation that is employed, the tablet precursor material is a blend of a tablet carrier material and protective material, where examples of these types of materials are provided above. The weight ratio of tablet carrier material to protective barrier material in this precursor blend may vary. In some instances, the weight ratio of these two types of materials in the precursor blends ranges from 0.5 to 80%, such as 1 to 50%, e.g., 10 to 40% protective material. During fabrication, following tablet pressing the resultant composition will be heated to a sufficient temperature to fuse the protective material of the tablet carrier and thereby seal the pores of the tablet. While the temperature to which the tablet is elevated during this fusing step may vary, in some instances this fusing temperature ranges from 25° C. to 160° C., such as 80° C. to 120° C. The duration at which the tablet is held at this fusing temperature is one sufficient for the protective material to melt and fill pores present in the tablet structure, and in some instances ranges from 0.1 to 4 hours, such as 5 to 60 min, including 10 to 30 min.

Another type of protective barrier of interest that includes two or more different materials is a barrier that is made up of a first protective barrier material component and a second solubilizer material of the first protective barrier component material. For example, a protective barrier material may be a lipid material, e.g., as described above. The second solubilizer material may be a component that enhances solubility of the lipid material upon contact with an aqueous medium, where examples of such lipid solubilizing materials include surfactants, e.g., as described above. The weight ratio of lipid material to solubilizer material may vary. In some instances, the weight ratio of these two types of materials ranges from 0.5% to 80%, such as 5% to 60% protective barrier material.

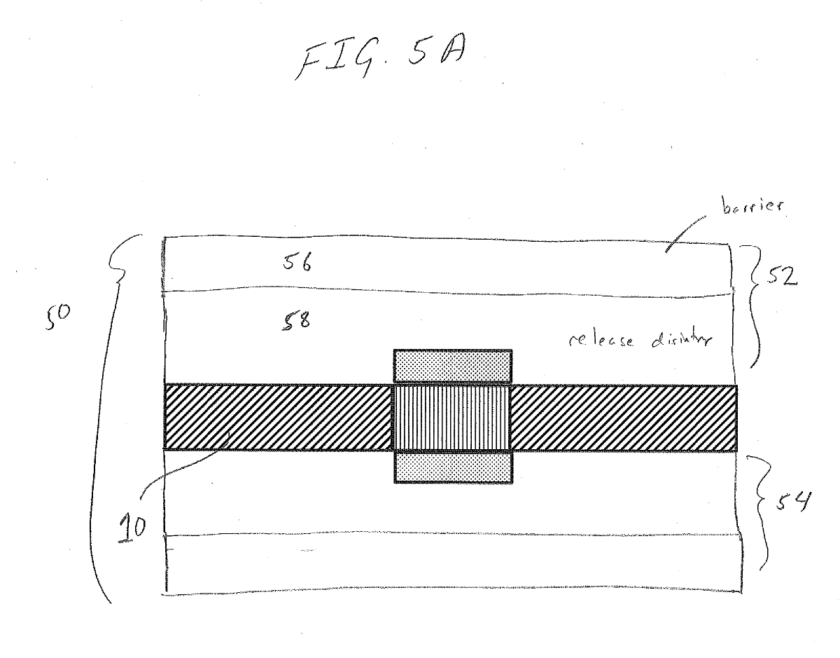

Instead of homogenous blend of two or more different materials, the protective layer may be a heterogeneous structure of two or more different materials, where regions (i.e., domains) of a second material, such as a water soluble material (e.g., a hydrogel, salt, etc.), are interspersed in regions of a hydrophobic material, e.g., a lipid material. FIG. 4 provides an illustration of such an ingestible composition. In FIG. 4, ingestible composition 40 includes IEM 1 position between two protective barriers, 42 and 44. Each of protective barriers 42 and 44 includes a first protective barrier material 46, e.g., as described above, and second regions or domains of a solubilizing material 48, e.g., as described above.

Protective barriers finding use as shelf-life stability components also include multilayer structures made up of two or more different materials. Of interest are multilayer structures of two or more materials, where the two materials may have differential properties that promote disruption of the protective barrier upon contact with a liquid. For example, the two or more distinct materials may exhibit different aqueous medium solubilities, such as one of the materials is more soluble in an aqueous medium than the other material. Alternatively, the two or more distinct materials may exhibit different aqueous medium physical properties, e.g., where one of the materials expands or shrinks in a manner different from the other upon contact with an aqueous medium, or where one of the materials produces gas upon contact with an aqueous medium, where the gas disrupts the barrier.

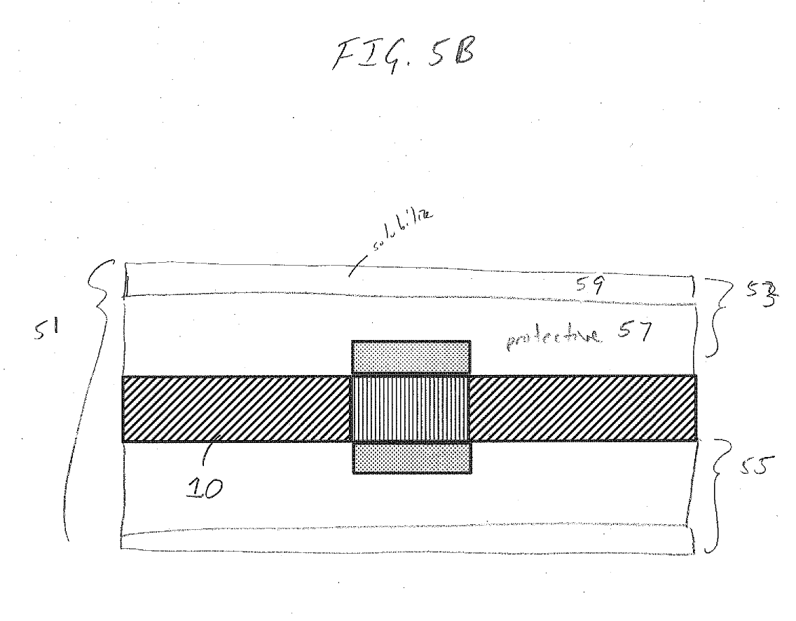

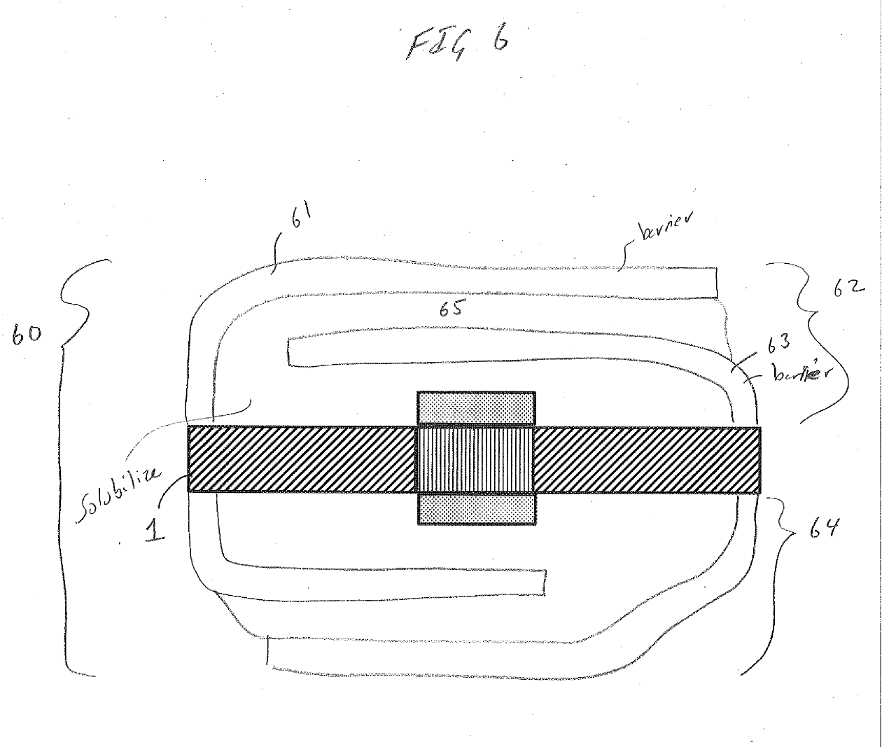

In one example of a multilayer protective barrier of interest, the multilayer structure is made up of a first layer of a protective barrier material and a second layer of a disrupting material that has a greater solubility in an aqueous medium than the first layer. An example of such a multilayer protective barrier is shown in the ingestible composition depicted in FIG. 5A. In FIG. 5A, ingestible composition 50 includes an IEM device 10 sandwiched between first and second multilayer protective barriers 52 and 54. Each multilayer protective barrier 52 and 54 is made up of a first layer 56 of a protective material, e.g., as described above, and a second layer 58 of a disrupting material that is more soluble in an aqueous medium that the protective material. Examples of disrupting materials that may make the second layer in such configurations include, but are not limited to: water-soluble polymers, e.g., water-soluble cellulosic materials, surfactants, salts, etc. Another example of a multilayer protective barrier is shown in the ingestible composition depicted in FIG. 5B. In FIG. 5B, ingestible composition 51 includes an IEM device 10 sandwiched between first and second multilayer protective barriers 53 and 55. Each multilayer protective barrier 53 and 55 is made up of a first layer 57 of a protective material, e.g., as described above, and a second layer 59 of a disrupting material that is more soluble that the protective material, e.g., as described above.

While the above examples were described in terms of the second material being more soluble that the protective material in an aqueous medium, as summarized above, other pairing of materials may also be employed. For example, the second disrupting material may have physical properties that differ from the protective material upon contact with the aqueous medium. Different physical properties may include water absorption, gas evolution, etc. For example, the second material may be a disrupting hydrogel which swells upon contact with an aqueous medium. Hydrogel materials of interest include, but are not limited to: pharmaceutically acceptable polymeric hydrogels, such as but not limited to: maltodextrin polymers comprising the formula (C₆H₁₂O₅)ₘ.H₂O, wherein m is 3 to 7,500, and the maltodextrin polymer comprises a 500 to 1,250,000 number-average molecular weight; a poly(alkylene oxide) represented by poly(ethylene oxide) and poly(propylene oxide) having a 50,000 to 750,000 weight-average molecular weight, e.g., by a poly(ethylene oxide) of at least one of 100,000, 200,000, 300,000, or 400,000 weight-average molecular weights; an alkali carboxyalkylcellulose, wherein the alkali is sodium, lithium, potassium or calcium, and alkyl is 1 to 5 carbons such as methyl, ethyl, propyl or butyl of 10,000 to 175,000 weight-average molecular weight; and a copolymer of ethylene-acrylic acid, including methacrylic and ethacrylic acid of 10,000 to 1,500,000 number-average molecular weight. Alternatively, the second disrupting material may be a material that is physiologically acceptable and produces a gas upon contact with an aqueous medium. Examples of such disrupting materials include materials that produce CO₂ upon contact with an aqueous medium, such as bicarbonate salts, e.g., sodium bicarbonate and potassium bicarbonate. In yet other embodiments, the second disrupting material may be a material that solubilizes the protective material, e.g., an enzyme that hydrolyzes the lipid protective material, such as described above.

Multilayer configurations of interest also include overlapping, e.g., inter-digitated, configurations, such as depicted in FIG. 6. In FIG. 6, ingestible composition 60 includes IEM device 10 sandwiched between first and second protective barriers 62 and 64. Each protective barrier 62 and 64 includes first and second overlapping barrier layers 61 and 63 of a protective material separated from each other by second disrupting material 65. In the configuration shown in FIG. 6, each barrier layer 61 and 63 is secured at one end to the edge of the skirt component of the IEM.

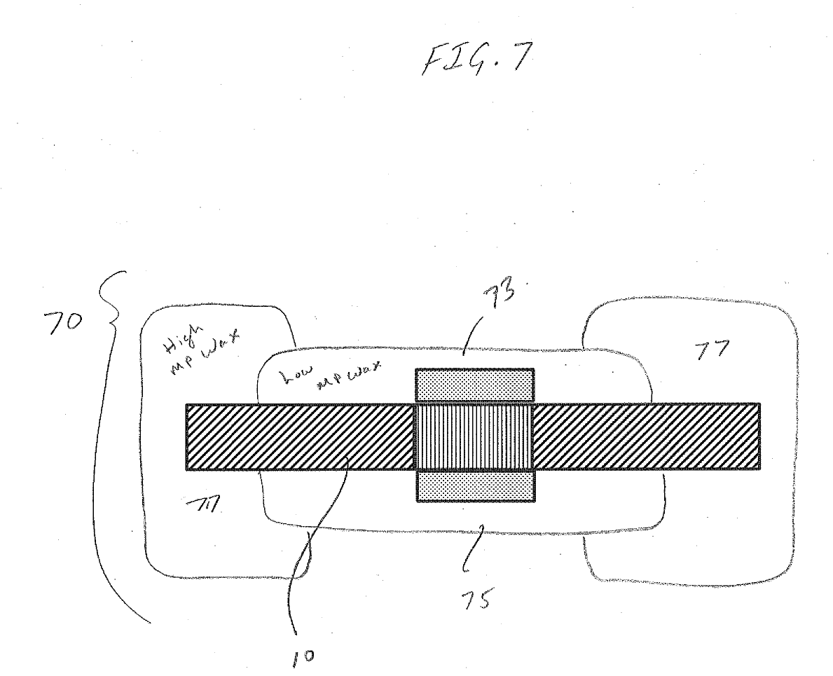

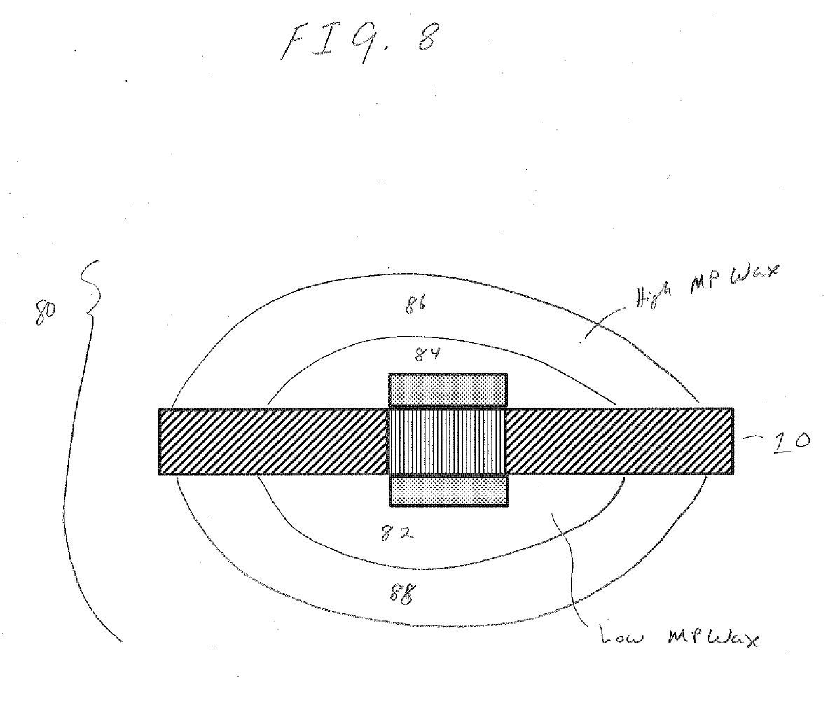

Another overlapping multilayer configuration is shown in FIG. 7. In FIG. 7, ingestible composition 70 includes IEM device 10 present between two opposing layers 73 and 75 of a first material. In addition, the edges of these opposing layers are capped with a second material 77. In composition 70, capping second material 77 has an annular configuration (e.g., having an outer diameter ranging from 5 mm to 8 mm and an inner diameter ranging from 2 mm to 5 mm) which partially overlaps the layers 73 and 75, and also caps the edge of the IEM skirt. In these configurations, the first and second materials may have different melting temperatures, e.g., the first material may have a melting temperature that is less than the melting temperature of the second material, and in some instances melts below 45° C. The differential in melting temperatures may vary, and in some instances ranges from 1 to 25° C., such as 2 to 20° C., including 5 to 15° C. Any convenient pairs of materials may be employed for the first and second materials, where the pairs of materials may be the same or different types of materials, e.g., a protective material and solubilizing material, two types of lipids having different melting points, etc. Specific material pairings of interest include, but are not limited to: low-melting point materials, such as low-melting point lipids (e.g., lipids that melt below 45° C.) and modified lipids/waxes; waxes and soluble polymers, and the like.

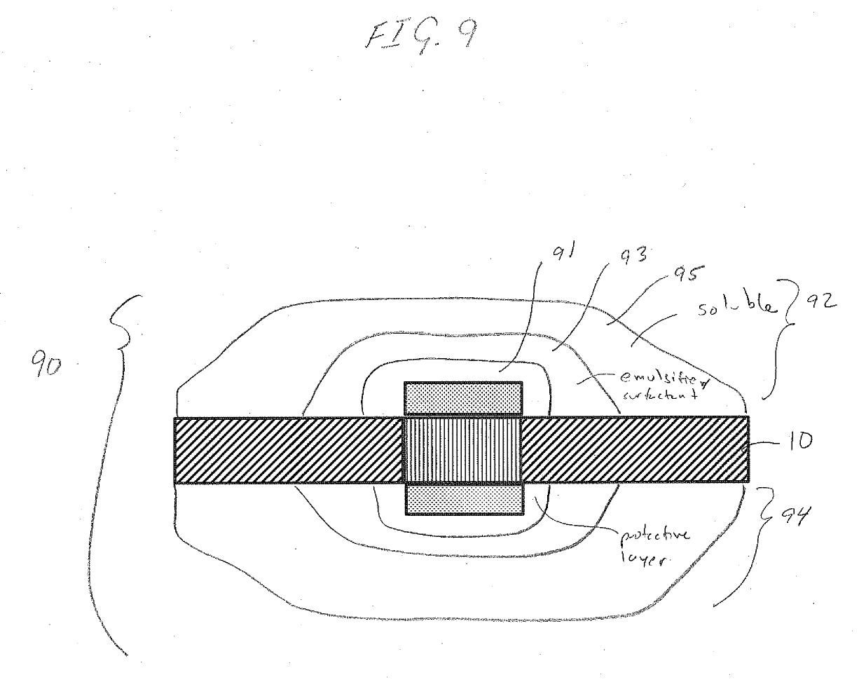

Yet another overlapping multilayer configuration is shown in FIG. 8. In FIG. 8, ingestible composition 80 includes IEM device 10 present between two opposing layers 84 and 82 of a first material. In addition, each of these opposing layers is further fully covered by second layers 86 and 88 made up of a second material. In these configurations, the first and second materials may have different melting temperatures, e.g., the first material may have a melting temperature that is less than the melting temperature of the second material. The differential in melting temperatures may vary, and in some instances ranges from 1 to 25° C., such as 2 to 20° C., including 5 to 15° C. Any convenient pairs of materials may be employed for the first and second materials, where the pairs of materials may be the same or different types of materials, e.g., a protective material and solubilizing material, two types of lipids having different melting points, etc.

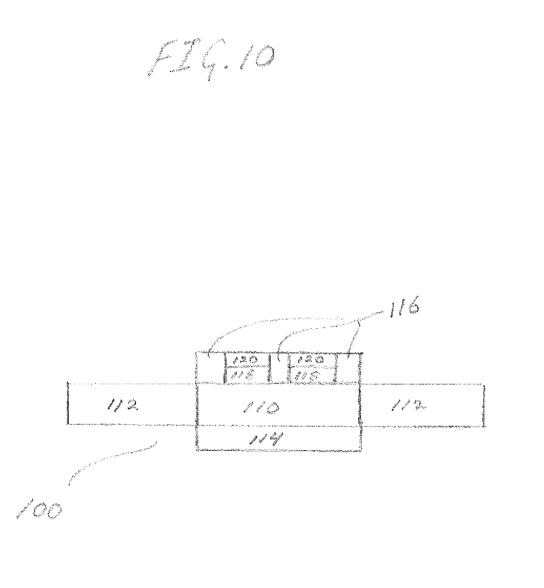

Yet another multilayer configuration showing protective barriers is depicted in FIG. 9. In FIG. 9, the ingestible composition is an example of compositions where the barrier is a multilayer structure of 2 or more distinct layers. In the particular embodiment depicted in FIG. 9, the protective barrier is made up of three layers. In FIG. 9, ingestible composition 90 includes IEM device 10 sandwiched between two protective barriers 92 and 94. Each protective barrier 92 and 94 includes three distinct layers: 91, 93 and 95. IEM proximal layer 91 is made up of a protective material, e.g., as described above. Intervening layer 93 comprises a protective layer solubilizing material, e.g., an enzyme, surfactant, etc. Outer layer 95 comprises a water soluble layer, such as HPMC, HPC, e.g., described above.

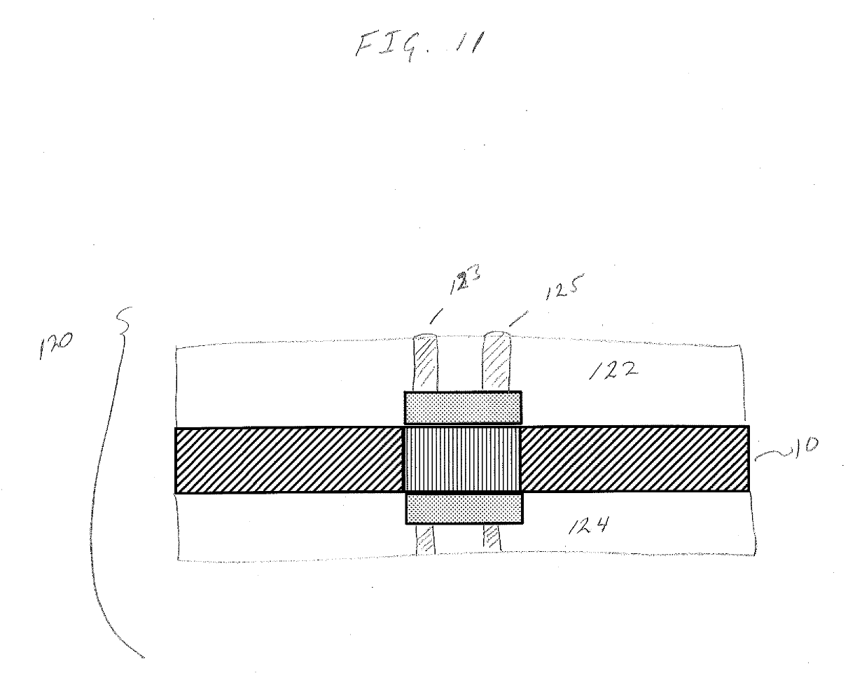

The protective barrier may also be a galvanic protective barrier. By “galvanic” is meant that the barrier material is one that is disrupted by galvanic corrosion upon immersion of the ingestible composition in a conducting fluid, e.g., stomach fluid. Galvanic protective barriers of interest include at least a protective metal. Protective metals of interest include those metals which are edible and have a water-sensitivity that is less than the sensitivity of the dissimilar material which they are intended to protect, e.g., CuCl. Specific protective metals of interest include magnesium, iron, copper, silver, etc. Where desired, a galvanic reaction initiator metal may be in contact with at least a portion of the protective metal, e.g., present along one or more edges (including the entire periphery of the protective metal), present in a region of the protective metal, etc. The galvanic reaction initiator metal is one that causes galvanic corrosion of the protective metal upon immersion in a conducting fluid, wherein galvanic reaction initiator metals of interest are ones that have a higher reduction potential than the protective metal. Examples of galvanic reaction initiator metals of interest include gold, platinum, etc. Any convenient configuration of the protective metal and the galvanic reaction initiator metal may be employed. FIG. 10 provides a view of an ingestible component that includes a galvanic protection layer according to one embodiment of the invention. In FIG. 10, IEM device 100 includes integrated circuit 110 and membrane 112. Also shown is second dissimilar material 114, e.g., magnesium, on the bottom side of integrated circuit 100. On the top side of integrated circuit 110 are two regions of first dissimilar material 118, e.g., CuCl. Separating the regions of first dissimilar material are walls of a galvanic reaction initiator metal 116, e.g., gold. Covering the layers of first dissimilar material 118 are metal protection layers 120, which are defect free layers that seal the first dissimilar material from the environment. The structure shown in FIG. 10 may be fabricated using any convenient protocol, e.g., by first forming wells on the top surface of integrated circuit 110 as defined by walls of the galvanic reaction initiator material, then depositing the first dissimilar material in the both of the wells and finally depositing the layer of protective metal over the layers of deposited first dissimilar material.

In some instances, the protective barrier is configured to provide aqueous liquid passage through the protective barrier upon contact of ingestible composition with an aqueous liquid. For example, the protective barrier may include one or more liquid passageways, which passageways may be filled (e.g., sealed (i.e., plugged)) with a material that readily dissolves upon contact with an aqueous liquid medium. An example of such an ingestible composition is depicted in FIG. 11. In FIG. 11, ingestible composition 120 includes IEM device 10 sandwiched between first and second protective barriers, 122 and 124. Protective barriers 122 and 124 each include liquid passageways 123 and 125. The diameter of the passageways may vary, ranging in some instances from 0.01 to 0.5 mm, such as 0.01 to 0.05 mm. The length of the passageways may also vary, ranging in some instances from 1 to 10 mm, such as 2 to 5 mm. The passageways may have a linear or non-linear configuration, as desired. The passageways may be filled with a material serves to seal the IEM device from a gaseous environment of the ingestible composition but that readily dissolves upon contact with an aqueous medium, thereby providing liquid access to the IEM device 10. Examples of such materials include any of the soluble materials listed above, e.g., salts, surfactants, etc. Where desired, such liquid passageways may be included in any of the protective barriers described above, e.g., as depicted in FIGS. 1 to 9.

In some instances, the protective barrier is configured to be disruptable by a device, e.g., an IEM device, present in the composition. For example, the protective barrier may include a material which melts in response to initial temperature changes produced upon IEM device initial activation, such that the initial IEM activation enhances the disruption of the protective barrier. Examples of such materials include, but are not limited to, low melting point lipids, e.g., and the like. Alternative, the protective barrier or a component thereof may be a material that is responsive (e.g., in terms of changing dimension) to a voltage change caused by the IEM device, where examples of such materials include conductive polymers, such as ionomers, e.g., sulfonated tetrafluoroethylene based fluoropolymer-copolymer.

Instead of or in addition to a protective barrier, e.g., as described above, the ingestible composition may include other types of water-vapor desensitizers. Other types of water-vapor desensitizers include water vapor sequestering materials, e.g., desiccants. A variety of different types of desiccant materials may be employed, where representative desiccant materials include solid materials, e.g., beads and strips or blocks of desiccant material, etc. Representative materials that may be employed as desiccants include, but are not limited to: molecular sieve, silica gel, CaSO₄, CaO, magnesium aluminum-metasilicate, and the like. Incorporated into the desiccant material may be an indicator that provides a detectable single, e.g., color change, that can be used to determine the remaining capacity of the desiccant, e.g., to determine whether or not a desiccant has reached capacity with respect to the amount of water that it can sequester. Indicator compounds of interest include, but are not limited to: CoCl₂ and the like.

Also of interest are barrier compositions that include an amount of a water/O₂ scavenger material. Examples of such materials include, but are not limited to: mercapto compounds, e.g., mercaptoalkanols, such as 3-mercapto-3-methyl-butan-1-ol, 3-mercapto-2-methyl-propan-1-ol and 2-Mercaptopyridine; BHA, BHT, benzothiazole, etc. When present, the amount of such compounds may vary, ranging in some instances from 1 ppb to 1%, such as 0.01% to 0.5%.

### Systems

Also provided are systems that include an ingestible device, e.g., an IEM, and a detection component, e.g., in the form of a receiver. Receivers of interest are those configured to detect, e.g., receive, a communication from an ingestible device, e.g., RFID ingestible device, IEM, etc. The signal detection component may vary significantly depending on the nature of the communication that is generated by the ingestible device. As such, the receiver may be configured to receive a variety of different types of signals, including but not limited to: RF signals, magnetic signals, conductive (near field) signals, acoustic signals, etc. In certain aspects, the receiver is configured to receive a signal conductively from an IEM, such that the two components use the body of the patient as a communication medium. As such, communication that is transferred between IEM and the receiver travels through the body, and requires the body as the conduction medium. The IEM communication may be transmitted through and received from the skin and other body tissues of the subject body in the form of electrical alternating current (a.c.) voltage signals that are conducted through the body tissues. This communication protocol has the advantage that the receivers may be adaptably arranged at any desired location on the body of the subject, whereby the receivers are automatically connected to the required electrical conductor for achieving the signal transmission, i.e., the signal transmission is carried out through the electrical conductor provided by the skin and other body tissues of the subject.

The receivers of interest include external, semi-implantable, and implantable receivers. In external aspects, the receiver is ex vivo, by which is meant that the receiver is present outside of the body during use. Examples include wearable patches, e.g., adhesive patches, torso bands, wrist(s) or arm bands, jewelry, apparel, mobile devices such as phones, attachments to mobile devices, etc. Where the receiver is implanted, the receiver is in vivo. Examples include cardiac can and leads, under-the-skin implants, etc. Semi-implantable devices include those designed to be partially implanted under the skin.

In certain aspects, the receiver may be configured to provide data associated with a received signal to a location external to said subject. For example, the receiver may be configured to provide data to an external data receiver, e.g., which may be in the form of a monitor (such as a bedside monitor), a computer, a personal digital assistant (PDA), phone, messaging device, smart phone, etc. The receiver may be configured to retransmit data of a received communication to the location external to said subject. Alternatively, the receiver may be configured to be interrogated by an external interrogation device to provide data of a received signal to an external location.

Receivers may be configured variously, e.g., with various signal receiving elements, such as electrodes, various integrated circuit components, one or more power components (such as power receivers or batteries), signal transmission components, housing components, etc.

In one aspect, for example, the receiver includes one or more of: a high power-low power module; an intermediary module; a power supply module configured to activate and deactivate one or more power supplies to a high power processing block; a serial peripheral interface bus connecting master and slave blocks; and a multi-purpose connector, as further described in PCT application serial No. PCT/US2009/068128 published as WO2010/075115, infra.

Receivers of interest include, but are not limited to, those receivers disclosed in: PCT application serial no. PCT/US2006/016370 published as WO 2006/116718; PCT application serial no. PCT/US2008/52845 published as WO 2008/095183; PCT application serial no. PCT/US2007/024225 published as WO 2008/063626; PCT application serial no. PCT/US2008/085048 published as WO 009/070773; PCT application serial no. PCT/US2009/068128 published as WO2010/075115; and U.S. provisional application Ser. No. 61/510,434 filed on Jul. 21, 2011 the disclosures of which applications (and particularly receiver components thereof) are herein incorporated by reference.

Systems of the invention may include an external device which is distinct from the receiver (which may be implanted or topically applied in certain aspects), where this external device provides a number of functionalities. Such an apparatus can include the capacity to provide feedback and appropriate clinical regulation to the patient. Such a device can take any of a number of forms. By example, the device can be configured to sit on the bed next to the patient, e.g., a bedside monitor. Other formats include, but are not limited to, PDAs, phones, such as smart phones, computers, etc. The device can read out the information described in more detail in other sections of the subject patent application, both from pharmaceutical ingestion reporting and from physiological sensing devices, such as is produced internally by a pacemaker device or a dedicated implant for detection of the pill. The purpose of the external apparatus is to get the data out of the patient and into an external device. One feature of the external apparatus is its ability to provide pharmacologic and physiologic information in a form that can be transmitted through a transmission medium, such as a telephone line, to a remote location such as a clinician or to a central monitoring agency.

### Manufacturing Methods

Also provided are methods of manufacturing ingestible compositions, e.g, as described herein. Aspects of the methods include combining an ingestible component (which may or may not include a device, such as an IEM) and a shelf-life stability component, e.g., as described above, in a manner sufficient to produce a shelf-life stable ingestible composition. Any convenient manufacturing protocol may be employed, where protocols of interest include both manual and automated protocols, as well as protocols that include both manual and automated steps. Protocols of interest that find use in various aspects of the fabrication methods described herein include lamination, molding, pressing, extrusion, stamping, coating (such as spray coating and dipping), etc. In some instances, fabrication protocols as described in PCT application serial nos. PCT/US2010/020142; PCT/US2006/016370 and PCT/US08/77753 (the disclosures of which are herein incorporated by reference) are employed.

Aspects of the fabrication protocols include stably associating the ingestible component with the shelf-life stability component. By “stably associating” is meant that the ingestible component and shelf-life stability component, e.g., protective barrier, do not separate from each other, at least until administered to the subject in need thereof, e.g., by ingestion. Any convenient approach for stably associating the ingestible component and the shelf-life stability component may be employed.

Where the ingestible component is positioned between two protective barrier components, e.g., as illustrated in FIGS. 2 to 5B, a protocol in which pre-fabricated protective barrier components may be employed. In such a protocol, the ingestible component may be positioned between the two pre-fabricated protective barrier components, e.g., in a manner sufficient to seal the ingestible component between the pre-fabricated protective barrier components. Where desired, an adhesive may be employed to secure the two protective barrier components together.

In a variation of the above protocol, a fabrication process may be one in which the protective barrier components are fabricated at the same time that the ingestible component is stably associated therewith. For example, a molding process may be employed where a protective barrier component precursor material, e.g., a liquid lipid/carrier material blend (such as described above), is positioned in a mold, followed by placement of an ingestible component (e.g., IEM) on the precursor material and then placement of an additional amount of precursor material on top of the ingestible component. Temperature modulation may be employed where appropriate, e.g., where the precursor material is a liquid at body temperature but a solid at room temperature. Following solidification of the precursor material, the resultant final product may be removed from the mold.

In yet another fabrication protocol of interest, a stamping protocol may be employed. For example, an ingestible component may be positioned between two sheets of a prefabricated multilayer protective barrier component, such as a sheet of a protective barrier component that includes a soluble layer and an insoluble layer, e.g., as described above. Once positioned between the two sheets, a stamping tool may be used to stamp and seal the two sheets around the ingestible component in a manner that encases the ingestible component in a sealed multilayer protective barrier. The stamping tool may be configuration to produce a product having any convenient shape, such as a disc, etc. Where desired, temperature modulation may be employed in such protocols.

In yet another fabrication protocol of interest, a coating process may be employed to stably associate the ingestible component with the shelf-life stability component. For example, a premade ingestible component in the form of a tablet may be provided, e.g., as described in in PCT application serial nos. PCT/US2010/020142; PCT/US2006/016370 and PCT/US08/77753 (the disclosures of which are herein incorporated by reference). This premade ingestible component may then be spray coated with a liquid protective barrier precursor material (e.g., as described above). Following spray coating, the coating material may be allowed to harden (e.g., by maintaining the coated tablet at a suitable temperature, such as room temperature) to produce the desired product.

Where desired, aspects of the above described or other suitable protocols may be combined to produce a fabrication protocol. For example, a molding process may be employed to make a product and the product spray coated with a further material, such as a soluble material.

### Methods of Use

Aspects of the invention further include methods of using the compositions, such as those described above. Aspects of such methods include administering an ingestible composition to a subject, e.g., by self-administration or via the assistance of another, such as a health care practitioner. Such methods may include placing the ingestible composition in the mouth of a subject such that the subject swallows the ingestible composition. In this manner, the subject ingests the ingestible composition. Ingestible compositions may be employed with a variety of subjects. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain aspects, the subjects will be humans.

Following ingestion, the methods may include receiving a signal emitted from an ingestible composition, such as an IEM comprising ingestible composition, e.g., at a receiver, such as described above. In some instances, the received signal is a conductively transmitted signal.

Ingestible composition may be employed in a variety of different applications. Applications of interest in which the ingestible composition comprises an IEM include, but are not limited to: monitoring patient compliance with prescribed therapeutic regimens; tailoring therapeutic regimens based on patient compliance; monitoring patient compliance in clinical trials; monitoring usage of controlled substances; monitoring the occurrence of a personal event of interest, such as the onset of symptoms, etc., and the like. Applications of interest are further described in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; and PCT application serial no. PCT/US2008/077753; the disclosures of which applications is herein incorporated by reference.

### Kits

Also provided are kits that include one or more ingestible compositions, such as described above. In those aspects having a plurality of ingestible compositions, the ingestible compositions may be packaged in a single container, e.g., a single tube, bottle, vial, and the like, or one or more dosage amounts may be individually packaged such that certain kits may have more than one container of ingestible compositions. In certain aspects the kits may also include a receiver, such as reviewed above. In certain aspects, the kits may also include an external monitor device, e.g., as described above, which may provide for communication with a remote location, e.g., a doctor's office, a central facility etc., which obtains and processes data obtained about the usage of the composition.

The subject kits may also include instructions for how to practice the subject methods using the components of the kit. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other aspects, the instructions are present as an electronic storage data file present on a suitable computer readable storage medium, e.g. CD-ROM, diskette, etc. In yet other aspects, the actual instructions are not present in the kit, but means for obtaining the instructions from a remote source, e.g. via the internet, are provided. An example of this aspect is a kit that includes a web address where the instructions can be viewed and/or from which the instructions can be downloaded. As with the instructions, this means for obtaining the instructions is recorded on a suitable substrate.

Some or all components of the subject kits may be packaged in suitable packaging to maintain sterility. In many aspects of the subject kits, the components of the kit are packaged in a kit containment element to make a single, easily handled unit, where the kit containment element, e.g., box or analogous structure, may or may not be an airtight container, e.g., to further preserve the sterility of some or all of the components of the kit.

It is to be understood that this invention is not limited to particular aspects described, as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

What is claimed is:

1. A composition comprising a shelf-life stability component physically associated with a minimally dimensioned component.

2. The composition according to claim 1, wherein the composition is an ingestible composition comprising the shelf-life stability component and an ingestible component physically associated with the ingestible composition.

3. The ingestible composition according to claim 2, wherein the composition further comprises an ingestible device.

4. The ingestible composition according to claim 3, wherein the ingestible device is an ingestible event marker.

5. The composition according to claim 1, wherein the ingestible composition is stable for 1 year or longer under conditions in which the temperature ranges from 10 to 40° C., the pressure ranges from 0.5 to 2.0 ATM and the relative humidity ranges from 10 to 100%.

6. The composition according to claim 1, wherein the shelf-life stability component comprises a water-vapor desensitizer.

7. The composition according to claim 5, wherein the water-vapor desensitizer comprises a protective barrier that rapidly disrupts upon contact with a liquid.

8. The composition according to claim 7, wherein the protective barrier comprises a homogeneous layer of a single material.

9. The composition according to claim 7, wherein the protective barrier comprises two or more distinct materials.

10. The composition according to claim 9, wherein the two or more distinct materials are present as a single homogeneous or heterogeneous layer.

11. The composition according to claim 9, wherein the two or more distinct materials are present as a multilayer structure.

12. The composition according to claim 9, wherein the two or more distinct materials exhibit different aqueous medium solubility.

13. The composition according to claim 9, wherein the two or more distinct materials exhibit different aqueous medium physical properties.

14. The composition according to claim 9, wherein the two or more distinct materials comprise a first material and a second material that solubilizes the first material.

15. The composition according to claim 7, wherein the protective barrier comprises a lipid.

16. The composition according to claim 7, wherein the protective barrier comprises a low-melting point material.

17. The composition according to claim 7, wherein the protective barrier is a galvanic protective barrier.

18. The composition according to claim 7, wherein the protective barrier is configured to be disruptable by a device present in the composition.

19. The composition according to claim 7, wherein the protective barrier is configured to provide aqueous liquid passage through the protective barrier upon contact of ingestible composition with an aqueous liquid.

20. The composition according to claim 7, wherein the protective barrier comprises a liquid passageway.

21. The composition according to claim 6, wherein the water-vapor desensitizer comprises a desiccant.

22. The composition according to claim 1, wherein minimally dimensioned component is a micro-battery.

23. A system comprising:
an ingestible composition comprising:
a shelf-life stability component; and
an ingestible component associated with the shelf-life stability component; and
a receiver configured to receive a communication associated with the ingestible composition.

24. A method comprising combining a minimally dimensioned component and a shelf-life stability component.

25. The method according to claim 24, wherein the minimally dimensioned component is an ingestible component and the method produces an ingestible composition.

26. The method according to claim 25, wherein the method comprises stably associating the ingestible component and the shelf-life stability component.

27. The method according to claim 26, wherein the method comprises one or more protocols selected from the group consisting of laminating, pressing, stamping, extruding, molding and coating.

28. The method according to claim 27, wherein at least a portion of the method is automated.
